(19) United States
(12) Patent Application Publication
(10) Pub. No.: US 2002/0034794 A1
(43) Pub. Date: Mar. 21, 2002

(54) Nucleotide sequences which encode the gpsA gene

(75) Inventors: Madhavan Nampoothiri, Kerala (IN); Brigitte Bathe, Salzkotten (DE); Lothar Eggeling, Juelich (DE); Hermann Sahm, Juelich (DE)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(73) Assignee: Degussa-Huels Aktiengesellschaft, Frankfurt am Main (DE)

(21) Appl. No.: 09/796,431

(22) Filed: Mar. 2, 2001

(30) Foreign Application Priority Data
Jul. 1, 2000 (DE) 100 32 174.7

(51) Int. Cl. C12P 13/08; C07H 21/04; C12N 1/21

(52) U.S. Cl. 435/115; 435/252.3; 536/23.2

(57) ABSTRACT

An isolated nucleic acid that encodes glycerol-3-phosphate dehydrogenase from coryneform bacteria, variants, homologs and fragments thereof. Hybridization probes and primers, vectors and host cells comprising such sequences. Coryneform bacterium with an enhanced ability to express glycerol-3-phosphate dehyrogenase. Methods of fermentative production of L-amino acids using coryneform bacteria having enhanced expression of glycerol-3-phosphate dehydrogenase.

### BACKGROUND OF THE INVENTION

1. Field of the Invention

The present invention relates to nucleotide sequences corresponding to the gpsA gene, which encode glycerol-3-phosphate dehydrogenase; to bacteria, such as coryneform bacteria, in which the expression or copy number of such genes is enhanced; and to processes for the fermentative production of L-amino acids, particularly L-glutamate or L-glutamic acid, using such bacteria.

2. Discussion of the Background

Amino acids, particularly L-lysine and L-glutamate, are used in human medicine, in the pharmaceutical industry, and in the food industry. They are also used for animal nutrition. Such amino acids may be produced by fermentation using certain strains of coryneform bacteria, particularly Corynebacterium glutamicum.

Attempts are continuously being made to improve the production process, due to the considerable importance of these amino acids. Process improvements can involve fermentation technology measures, such as stirring and supplying a culture with oxygen; can relate to the composition of the culture media, such as the sugar concentration during fermentation; to work-up or purification conditions used to provide the desired form of product, for example, by use of various purification methods such as ion exchange chromatography; or to the modification of the intrinsic production properties of the microorganism itself.

To improve the intrinsic production properties of a microorganism, methods of mutagenesis, selection and mutant screening are often employed. In this manner, strains are obtained which are resistant to antimetabolites, such as the lysine analogon S-(2-aminoethyl)-cysteine, or which are auxotrophic for metabolites of regulatory importance, and which produce L-amino acids such as L-lysine or L-glutamate/L-glutamic acid.

Moreover, for some years methods of recombinant DNA technology have been used to improve strains of Corynebacterium which produce amino acids. This has been achieved by amplifying individual amino acid biosynthesis genes and investigating the effect on amino acid production. Review articles on this topic, amongst other sources, are those by Kinoshita (“Glutamic Acid Bacteria”, in: Biology of Industrial Microorganisms, Demain and Solomon (Eds.), Benjamin Cummings, London, UK, 1985, 115-142), Hilliger (BioTec 2, 40-44 (1991)), Eggeling (Amino Acids 6:261-272 (1994)), Jetten and Sinskey (Critical Reviews in Biotechnology 15, 73-103 (1995)) and Sahm et al. (Annals of the New York Academy of Science 782, 25-39 (1996)).

However, there remains a critical need for improved methods of producing amino acids and thus for the provision of strains of bacteria producing higher amounts of amino acids. On a commericial or industrial scale even small improvements in the yield of amino acids, or the efficiency of their production, are economically significant. Metabolic pathways, as well as their regulation are complex and prior to the present invention, it was not recognized that enhancement or over-expression of the gpsA gene, encoding glycerol-3-phosphate dehydrogenase, would improve amino acid yields.

### SUMMARY OF THE INVENTION

One object of the present invention, is providing a new process adjuvant for improving the fermentative production of amino acids, particularly L-lysine and L-glutamate/L-glutamic acid. Such process adjuvants include enhanced bacteria, preferably enhanced coryneform bacteria which express high amounts of glycerol-3-phosphate dehydrogenase which is encoded by the gpsA gene.

Thus, another object of the present invention is providing such an enhanced bacterium, which expresses an enhanced amount of glycerol-3-phosphate dehydrogenase or gene products of the gpsA gene.

Another object of the present invention is providing a bacterium, preferably a coryneform bacterium, which expresses a polypeptide that has an enhanced glycerol-3-phosphate activity or an enhanced stability.

Another object of the invention is to provide a nucleotide sequence encoding a polypeptide which has glycerol-3-phosphate dehydrogenase activity, and replicable nucleic acids, vectors and host cells comprising this sequence. One embodiment of such a sequence is the nucleotide sequence of SEQ ID NO: 1.

A further object of the invention is a method of making glycerol-3-phosphate dehydrogenase or an isolated polypeptide having a glycerol-3-phosphate polypeptide activity, as well as use of such isolated polypeptides in the production of amino acids. One embodiment of such a polypeptide is the polypeptide having the amino acid sequence of SEQ ID NO: 2.

Other objects of the invention include methods of detecting nucleic acid sequences homologous to SEQ ID NO: 1, particularly nucleic acid sequences encoding polypeptides that have a glycerol-3-phosphate activity, and methods of making nucleic acids encoding such polypeptides.

The above objects highlight certain aspects of the invention. Additional objects, aspects and embodiments of the invention are found in the following detailed description of the invention.

### BRIEF DESCRIPTION OF THE DRAWINGS

FIG. 1 shows a partial restriction map of Plasmid pJC1 gpsA.

### DETAILED DESCRIPTION OF THE INVENTION

Amino acids, particularly L-lysine and L-glutamate/L-glutamic acid, are used in human medicine, in veterinary medicine, in the pharmaceutical industry and particularly in the food industry. There is therefore a general interest in the provision of new, improved processes for producing amino acids, particularly L-glutamate/L-glutamic acid.

When L-lysine or lysine or L-glutamate/L-glutamic acid or glutamate are mentioned below, this refers not only to the bases of these amino acids, but also the salts thereof. For instance, monosodium glutamate is a salt of glutamic acid. Salts of these amino acids may be produced by conventional chemical methods. The term “L-glutamate” includes glutamic acid as well as salts of glutamic acid.

The present invention relates to a bacterium, preferably a coryneform bacterium, comprising an enhanced gpsA gene or an enhanced gene encoding a polypeptide having a glycerol-3-phosphate dehydrogenase activity.

In this connection, the terms “enhanced” or “enhancement” mean increasing the quantity, stability or intracellular activity of one or more enzymes in a microorganism which are encoded by the corresponding DNA, such as the gpsA gene or its homologs.

Enhancement can be achieved with the aid of various manipulations of the bacterial cell.

In order to achieve enhancement, particularly over-expression, the number of copies of the corresponding gene can be increased. Preferably the average copy number of the gene of interest is increased to 2-times the normal number, more preferably at least 3, 4 or 5-times the normal copy number, most preferably at least 6-10 times the normal copy number.

Additionally, a strong promoter can be used, or the promoter-and regulation region or the ribosome binding site which is situated upstream of the structural gene can be engineered or mutated. Expression cassettes which are incorporated upstream of the structural gene act in the same manner.

Similarly, it is possible to increase gene expression in the course of the fermentative production of an amino acid, such as L-lysine- or L-glutamate/L-glutamic acid by employing inducible promoters.

A gene can also be used which encodes a corresponding or variant enzyme with a high activity. Preferably the corresponding enzyme has a greater activity than the native form of the enzyme, more preferably at least in the range of 5, 10, 25% or 50% more activity, most preferably more than twice the activity of the native enzyme.

Expression can also be improved by measures for extending the life of the m-RNA.

Furthermore, enzyme activity as a whole can be increased by preventing the degradation of the expressed enzyme. Moreover, these measures can optionally be combined in any desired manner.

The microorganisms to which the present invention relates produce L-amino acids, particularly L-lysine and L-glutamate/L-glutamic acid, from glucose, saccharose, lactose, fructose, maltose, molasses, starch or cellulose, or from glycerol and ethanol. They can be representatives of coryneform bacteria, particularly of the genus Corynebacterium. A bacterium of the genus Corynebacterium which should be mentioned in particular is the Corynebacterium glutamicum species, which is known to those skilled in the art for its capacity of producing L-amino acids.

Examples of suitable strains of the genus Corynebacterium, particularly of the Corynebacterium glutamicum species, are the known wild-type strains:

Corynebacterium glutamicum ATCC13032

Corynebacterium acetoglutamicum ATCC15806

Corynebacterium acetoacidophilum ATCC13870

Corynebacterium thermoaminogenes FERM BP-1539

Corynebacterium melassecola ATCC17965

Brevibacterium flavum ATCC14067

Brevibacterium lactofermentum ATCC13869, and

Brevibacterium divaricatum ATCC14020, and L-lysine-producing mutants or strains which are produced therefrom, such as:

Corynebacterium glutamicum FERM-P 1709

Brevibacterium flavum FERM-P 1708

Brevibacterium lactofermentum FERM-P 1712

Corynebacterium glutamicum FERM-P 6463

Corynebacterium glutamicum FERM-P 6464, and

Corynebacterium glutamicum DSM5715.

Preferably, a bacterial strain enhanced for expression of a gpsA-like gene that encodes a polypeptide with glycerol-3-phosphate dehydrogenase activity, will improve amino acid yields at least 1%, more preferably from 2-5%, and most preferably at least 5%-100%.

The present invention further relates to a polynucleotide which may be isolated from a bacterium, such as a coryneform bacteria, containing a polynucleotide sequence selected from the group comprising

a) a polynucleotide, at least 70% of which is identical, similar or homologous with a polynucleotide which encodes a polypeptide which contains the amino acid sequence of SEQ ID No.2,

b) a polynucleotide which encodes a polypeptide which comprises an amino acid sequence, at least 70% of which is identical, similar or homologous with the amino acid sequence of SEQ ID No. 2,

c) a polynucleotide which is complementary to the polynucleotides of a) or b), and

d) a polynucleotide containing at least 15 successive nucleotides of the polynucleotide sequence of a), b) or c).

In the context of the present Application, a polynucleotide sequence is “homologous” with or “similar” to the sequence according to the invention if at least 70%, preferably at least 80%, most preferably at least 90% of its base composition and base sequence corresponds or has identity to the sequence according to the invention.

According to the invention, a “homologous protein” is to be understood to comprise a protein which contains an amino acid sequence at least 70% of which, preferably at least 80% of which, most preferably at least 90% of which, corresponds or has identity to the amino acid sequence which is encoded by the gpsA gene (SEQ ID No.1), wherein “corresponds” is to be understood to mean that the corresponding amino acids are either identical or are mutually homologous amino acids. The expression “homologous amino acids” denotes those which have corresponding properties, particularly with regard to their charge, hydrophobic character, steric properties, etc.

Homology, sequence similarity or sequence identity of nucleotide or amino acid sequences may be determined conventionally by using known software or computer programs such as the BestFit or Gap pairwise comparison programs (GCG Wisconsin Package, Genetics Computer Group, 575 Science Drive, Madison, Wis. 53711). BestFit uses the local homology algorithm of Smith and Waterman, Advances in Applied Mathematics 2: 482-489 (1981), to find the best segment of identity or similarity between two sequences. Gap performs global alignments: all of one sequence with all of another similar sequence using the method of Needleman and Wunsch, J. Mol. Biol. 48:443-453 (1970). When using a sequence alignment program such as BestFit, to determine the degree of sequence homology, similarity or identity, the default setting may be used, or an appropriate scoring matrix may be selected to optimize identity, similarity or homology scores. Similarly, when using a program such as BestFit to determine sequence identity, similarity or homology between two different amino acid sequences, the default settings may be used, or an appropriate scoring matrix, such as blosum45 or blosum80, may be selected to optimize identity, similarity or homology scores.

The present invention also encompasses polynucleotides that hybridize, preferably under stringent conditions, to the nucleotide sequence of SEQ ID NO: 1, or its complement. Hybridization procedures as well known in the art and instructions for identifying DNA sequences by means of hybridization can be found by the expert, inter alia, in the hand book “The DIG System User's Guide for Filter Hybridization” from Boehringer Mannheim GmbH (Mannheim, Germany, 1993) or in Liebl et al., International Journal of Systematic Bacteriology 41:255-260 (1991). The hybridization may take place under stringent conditions, that is to say only hybrids in which the probe and target sequence, i.e. the polynucleotides contacted with the probe, are at least 70% identical are formed. It is known that the stringency of the hybridization, including the washing steps, is influenced or determined by varying the buffer composition, the temperature, and the salt concentration. The hybridization reaction is preferably carried out under a relatively low stringency compared with the washing steps (Hybaid Hybridization Guide, Hybaid Limited, Teddington, UK, 1996).

For example, a 5×SSC buffer at a temperature of about 50° C. to 68° C. can be employed for the hybridization reaction. Probes can also hybridize here with polynucleotides which are less than 70% identical to the sequence of the probe. Such hybrids are less stable and are removed by washing under stringent conditions. This can be achieved, for example, by washing at a salt concentration of 2×SSC and optionally again at 0.5×SSC (The DIG System User's Guide for Filter Hybridization, Boehringer Mannheim, Mannheim, Germany, 1995), and the temperature can be raised to 50° C. to 68°C. during washing. For higher stringency, it is also possible to lower the salt concentration to 0.1×SSC. Polynucleotide fragments which are, for example, at least 70%, at least 80%, at least 90-95%, or at least 96-99% identical to the sequence of the probe employed can be isolated by increasing the hybridization temperature stepwise from 50° C. to 68° C. in steps of approximately 1° C. to 2° C. It is also possible to isolate polynucleotide fragments which are completely identical to the sequence of the probe employed. Further instructions on hybridization procedures and conditions are publicly and commercially available and are obtainable in the form of commericial kits, e.g. DIG Easy Hyb from Roche Diagnostics, GmbH, Mannheim, Germany, Catalogue No. 1603558.

The present invention also relates to a polynucleotide as described above, which is preferably a replicable DNA containing:

(i) the nucleotide sequence shown in SEQ ID No. 1, or

(ii) at least one sequence which corresponds to sequence (i) in the context of the degeneration of the genetic code, or

(iii) at least one sequence which hybridizes with the sequence complementary to sequence(i) or (ii),

(iv) at least one sequence that has at least 70% sequence homology, similarity or identity with the sequence shown in SEQ ID No. 1,

(v) functionally neutral mutations in (i) which result in the same or a homologous amino acid.

(vi) a fragment of the nucleotide sequence of (i), (ii), (iii), (iv) or (v), which encodes a polypeptide having glycerol-3-phosphate dehydrogenase activity.

The present invention further relates to: a replicable polynucleotide which comprises or consists of the nucleotide sequence of SEQ ID No. 1,

a polynucleotide sequence which encodes a polypeptide which comprises or consists of the amino acid sequence of SEQ ID No. 2,

a vector containing the DNA sequence of C. glutamicum which encodes the gpsA gene, contained in the vector (plasmid) pJC1 gpsA deposited as a Corynebacterium glutamicum with the number DSM 13493,

and coryneform bacteria which serve as host cells and which contain the above-mentioned vector or in which the gpsA gene is enhanced.

The present invention also relates to polynucleotides which contain the complete gene with the polynucleotide sequence corresponding to SEQ ID No. 1 or fragments thereof, and which can be obtained by screening by means of the hybridization of a corresponding gene bank with a probe which contains the sequence of said polynucleotide corresponding to SEQ ID No. 1 or a fragment thereof, and isolation of the desired DNA sequence.

Polynucleotide sequences according to the invention are suitable as hybridization probes for RNA, cDNA and DNA, in order to isolate the complete length of cDNA which encodes glycerol-3-phosphate dehydrogenase and in order to isolate those cDNAs or genes which exhibit a high degree of similarity to the sequence of the glycerol-3-phosphate dehydrogenase gene.

Polynucleotide sequences according to the invention are also suitable as primers for polymerase chain reaction (PCR) for the production of DNA which encodes glycerol-3-phosphate dehydrogenase.

Oligonucleotides such as these, which serve as probes or primers, can contain more than 30, preferably up to 30, more preferably up to 20, most preferably at least 15 successive nucleotides. Oligonucleotides with a length of at least 40 or 50 nucleotides are also suitable.

The term “isolated” means separated from its natural environment.

The term “polynucleotide” refers in general to polyribonucleotides and polydeoxyribonucleotides, and can denote an unmodified RNA or DNA or a modified RNA or DNA.

The term “polypeptides” is to be understood to mean peptides or proteins which contain two or more amino acids which are bound via peptide bonds.

The term “fragment” is to be understood to refer to polypeptides or amino acid sequences shorter than the polypeptide of SEQ ID No. 2, preferably having a glycerol-3-dehydrogenase activity. Chimeric or fusion proteins may comprise a fragment of SEQ ID No. 2 or a protein similar or homologous to SEQ ID No. 2 having glycerol-3-phosphate activity and exogenous amino acid residues. Methods of engineering fragments using recombinant DNA techniques, protein engineering techniques, such as proteolytic digestion, or by protein synthesis are well known in the art.

The polypeptides according to invention include polypeptides corresponding to SEQ ID No. 2, particularly those with a biological activity of glycerol-3-phosphate dehydrogenase, and also include those with sequences, at least 70% of which, preferably at least 80% of which, are homologous, identical or similar to a polypeptide corresponding to SEQ ID No. 2, and most preferably those which exhibit a homology, identity or similarity of least 90% to 95% to 99% with the polypeptide corresponding to SEQ ID No. 2 and which have a glycerol-3-phosphate dehydrogenase activity.

The term “variant” or “mutant” polypeptides is to be understood to mean a peptide or polypeptide which is at least 70%, preferably at least 80%, most preferably at least 90% homologous, similar or identical to SEQ ID No. 2. It also refers to peptides or polypeptides which are produced by nucleic acids which hydridize, preferably under stringent conditions, to the nucleotide sequence of SEQ ID No. 1. Such peptides may comprise deletions, insertions, substitutions, or transpositions of amino acid residues. Most preferably, a variant or mutant peptide or polypeptide has glycerol-3-phosphate dehydrogenase activity.

The variant polypeptides of the present invention having glycerol-3-dehydrogenase activity and homology or similarity to SEQ ID NO. 2 may be produced by conventional mutagenesis or directed evolution procedures. Random mutagenesis of the nucleotide sequence of SEQ ID No. 1 can be accomplished by several different techniques known in the art, such as by chemical mutagenesis using agents such as nitrosoguanidine, UV or X-ray irradiation, insertion of an oligonucleotide linker randomly into a plasmid comprising SEQ ID No. 1, or by techniques such as error-prone PCR mutagenesis. For example, such techniques can be used to generate a library of plasmids containing variants of SEQ ID No. 1. These variants include those which encode proteins with substitution, deletion, addition or transposition of one or more amino acid residues of SEQ ID No. 2.

A plasmid library expressing such variant proteins may be screened by conventional means for clones having a particular degree of similarity to SEQ ID No.1 or for expression of proteins having glycerol-3-phosphate dehydrogenase activity or for an ability to enhance amino acid production.

The invention also relates to a process for the fermentative production of L-amino acids, particularly L-lysine and L-glutamate/L-glutamic acid, using bacteria, such as coryneform bacteria, preferably those which already produce an amino acid and in which the nucleotide sequences which encode the gpsA gene are enhanced, or in particular are over-expressed.

In the present invention, the gpsA gene of C. glutamicum which encodes glycerol-3-phosphate dehydrogenase (EC 1.1.1.94) is demonstrated for the first time.

In order to isolate a gpsA-like gene or other genes of bacteria, such as C. glutamicum, a gene bank of this microorganism (or a related microorganism) is first constructed in E. coli. The construction of gene banks is described in generally known textbooks and handbooks. Examples thereof include the textbook by Winnacker: Gene und Klone, Eine Einführung in die Gentechnologie (Verlag Chemie, Weinheim, Germany, 1990) or the Handbook by Sambrook et al.: Molecular Cloning, A Laboratory Manual (Cold Spring Harbor Laboratory Press, 1989). One very well known gene bank is that of the E. coli K-12 strain W3110, which was constructed by Kohara et al. (Cell 50, 495-508 (1987)) in λ-vectors. Bathe et al. (Molecular and General Genetics, 252:255-265, 1996) described a gene bank of C. glutamicum ATCC13032, which with the aid of the cosmid vector SuperCos I (Wahl et al., 1987, Proceedings of the National Academy of Sciences USA, 84:2160-2164) was constructed in the E. coli K-12 strain NM554 (Raleigh et al., 1988, Nucleic Acids Research 16:1563-1575. Bormann et al. (Molecular Microbiology 6(3), 317-326 (1992)) in turn describe a gene bank of C. glutaiicum ATCC13032 using the cosmid pHC79 (Hohn and Collins, Gene 11, 291-298 (1980)). Plasmids such as pBR322 (Bolivar, Life Sciences, 25, 807-818 (1979)) or pUC9 (Vieira et al., 1982, Gene, 19:259-268) can also be used for the production of a gene bank of C. glutamicum in E. coli. Those E. coli strains which are restriction-and recombination-deficient are particularly suitable as hosts. One example thereof is the strain DH5αmcr, which was described by Grant et al. (Proceedings of the National Academy of Sciences USA, 87 (1990) 4645-4649). Long DNA fragments which are cloned with the aid of cosmids can subsequently again be subcloned in common vectors which are suitable for sequencing and can then be sequenced, as described by Sanger et al. (Proceedings of the National Academy of Sciences of the United States of America, 74:5463-5467, 1977).

In this manner, a new DNA sequence of C. glutamicum has been obtained which comprises the gpsA gene and encodes gpsA gene products, and which as SEQ ID No. 1 forms part of the present invention. Moreover, the amino acid sequence of the corresponding protein has been derived from the present DNA sequence using the methods described above. The resulting amino acid sequence of the gpsA gene product is illustrated in SEQ ID No. 2. Fragments of this or similar sequences having biological activity, such as a glycerol-3-phosphate dehydrogenase activity, may also be produced based on the above information.

The invention also relates to coding DNA sequences which result from SEQ ID No. 1 by degeneration of the genetic code. In the same manner, the invention further relates to DNA sequences which hybridize with SEQ ID No. 1 or with parts of SEQ ID No. 1.

Moreover, one skilled in the art is also aware of conservative amino acid replacements such as the replacement of glycine by alanine or of aspartic acid by glutamic acid in proteins as “sense mutations” which do not result in a fundamental change in the activity of the protein, i.e. which are functionally neutral. It is also known that changes at the N-and/or terminus of a protein do not substantially impair the function thereof, and may even stabilise said function. Amongst other sources, one skilled in the art will find information on this topic in the articles by Ben-Bassat et al. (Journal of Bacteriology 169:751-757 (1987)), by O'Regan et al. (Gene 77:237-251 (1989)), by Sahin-Toth et al. (Protein Sciences 3:240-247 (1994)), by Hochuli et al. (Bio/Technology 6:1321 -1325 (1988)) and in known textbooks on genetics and molecular biology. The present invention also relates to amino acid sequences which result in a corresponding manner from SEQ ID No. 2.

In the same manner, the present invention also relates to DNA sequences which hybridize with SEQ ID No. 1 or with parts of SEQ ID No. 1. Finally, the present invention relates to DNA sequences which are produced by polymerase chain reaction (PCR) using oligonucleotide primers which result from SEQ ID No. 1. Oligonucleotides of this type typically have a length of at least 15 nucleotides.

Amongst other sources, one skilled in the art will find instructions for the identification of DNA sequences by means of hybridization in the Handbook “The DIG System User's Guide for Filter Hybridization” published by Boehringer Mannheim GmbH (Mannheim, Germany, 1993) and in the article by Liebl et al. (International Journal of Systematic Bacteriology (1991) 41: 255-260). Amongst other sources, one skilled in the art will find instructions for the amplification of DNA sequences with the aid of polymerase chain reaction (PCR) in the Handbooks by Gait: Oligonucleotides synthesis: a practical approach (IRL Press, Oxford, UK, 1984) and by Newton and Graham: PCR (Spektrum Akademischer Verlag, Heidelberg, Germany, 1994).

The work which has been carried out on the present invention has enabled it to be ascertained that, after enhancement of their gpsA gene has been effected, coryneform bacteria produce amino acids, particularly L-lysine and L-glutamate/L-glutamic acid, in an improved manner.

The genes or gene constructs concerned can either be present with different numbers of copies in plasmids, or can be integrated and amplified in the chromosome. Alternatively, over-expression of the gene concerned can be effected by changing the composition of the medium and by changing the way in which cultivation is effected.

Amongst other sources, one skilled in the art will find instructions on this topic in the articles by Martin et al. (Bio/Technology 5, 137-146 (1987)), by Guerrero et al. (Gene 138, 35-41 (1994)), by Tsuchiya and Morinaga (Bio/Technology 6, 428-430 (1988)), by Eikmanns et al. (Gene 102, 93-98 (1991)), in European Pat. Specification EPS 0 472 869, in U.S. Pat. No. 4,601,893, in the articles by Schwarzer and Pühler (Bio/Technology 9, 84-87 (1991), by Reinscheid et al. (Applied and Environmental Microbiology 60, 126-132 (1994)), by LaBarre et al. (Journal of Bacteriology 175, 1001-1007 (1993)), in Patent Application WO 96/15246, in the article by Malumbres et al. (Gene 134, 15 -24 (1993)), in Japanese laid-open Patent Specification JP-A-10-229891, in the articles by Jensen and Hammer (Biotechnology and Bioengineering 58, 191-195 (1998)), by Makrides (Microbiological Reviews 60:512-538 (1996)) and in known textbooks on genetics and molecular biology.

For example, the gpsA gene according to the invention has been over-expressed with the aid of plasmids.

Suitable plasmids are those which are replicated and expressed in coryneform bacteria. Numerous known plasmid vectors such as pZl (Menkel et al., Applied and Environmental Microbiology (1989) 64: 549-554), pEKEx1 (Eikmanns et al., Gene 102:93-98 (1991)) or pHS2-1 (Sonnen et al., Gene 107:69-74 (1991)) are based on the cryptic plasmids pHM1519, pBL1 or pGA1. Other plasmid vectors, such as those which are based on pCG4 (U.S.-A 4,489,160), or pNG2 (Serwold-Davis et al., FEMS Microbiology Letters 66, 119-124 (1990)), or pAG1 (U.S.-A 5,158,891), can be used in the same manner.

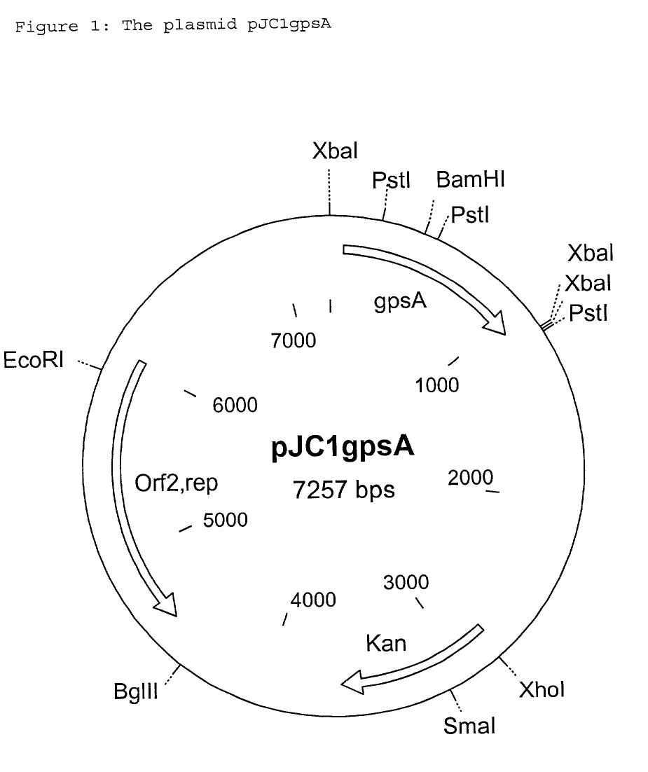

One example of a plasmid with the aid of which the gpsA gene can be over-expressed is pJC1gpsA (FIG. 1), which is based on the E. coli-C. glutamicum shuttle vector pJC1 (Cremer et al., 1990, Molecular and General Genetics 220: 478-480) and which contains the DNA sequence of C. glutamicum which encodes the gpsA gene. This is contained in the strain DSM5715/pJC1gpsA.

Also suitable are those plasmid vectors by means of which the process of gene amplification by integration in the chromosome can be employed, such as that described, for example, by Reinscheid et al. (Applied and Environmental Microbiology 60, 126-132 (1994)) for the duplication or amplification of the hom-thrb operon. In this method, the complete gene is cloned in a plasmid vector which can replicate in a host (typically E. coli), but which cannot replicate in C. glutamicum. Examples of suitable vectors include pSUP301 (Simon et al., Bio/Technology 1, 784-791 (1983)), pK18mob or pK19mob (Schäfer et al., Gene 145, 69-73 (1994)), pGEM-T (Promega Corporation, Madison, WI, U.S.A), pCR2.1-TOPO (Shuman (1994). Journal of Biological Chemistry 269:32678-84; U.S.-A 5,487,993), pCR®Blunt (Invitrogen, Groningen, Holland; Bernard et al., Journal of Molecular Biology, 234: 534-541 (1993)) or pEM1 (Schrumpf et al, 1991, Journal of Bacteriology 173:4510-4516). The plasmid vector which contains the gene to be amplified is subsequently converted by conjugation or transformation into the desired strain of C. glutamicum. The conjugation method is described, for example, by Schäfer et al. (Applied and Environmental Microbiology 60, 756-759 (1994)). Transformation methods are described, for example, by Thierbach et al. (Applied Microbiology and Biotechnology 29, 356-362 (1988)), by Dunican and Shivnan (Bio/Technology 7, 1067-1070 (1989)) and by Tauch et al. (FEMS Microbiological Letters 123, 343-347 (1994)). After homologous recombination by means of a “cross over” occurrence, the resulting strain contains at least two copies of the gene concerned.

Moreover, apart from the gpsA gene, it may be advantageous for the production of amino acids, particularly L-glutamate/L-glutamic acid, to intensify or over-express one or more genes which encode enzymes of the biosynthesis route employed, of glycolysis, of anaplerosis, of the citric acid cycle or of amino acid export.

Thus, for the production of L-lysine, for example, one or more genes selected from the following group can be simultaneously enhanced, and in particular can be over-expressed or amplified:

the dapA gene which encodes dihydrodipicolinate synthase (EP-B 0 197 325), or

the dapE gene which encodes succinyl diaminopimelate desuccinylase, or

the lysc gene which encodes feed-back resistant aspartate kinase (Kalinowski et al. (1990), Molecular and General Genetics 224, 317-324), or

the gap gene which encodes glyceraldehyde-3-phosphate dehydrogenase (Eikmanns (1992), Journal of Bacteriology 174:6076-6086), or

the tpi gene which encodes triose phosphate isomerase (Eikmanns (1992), Journal of Bacteriology 174:6076-6086), or

the pgk gene which encodes 3-phosphoglycerate kinase (Eikmanns (1992), Journal of Bacteriology 174:6076-6086), or

the pyc gene which encodes pyruvate carboxylase (DE-A-19831609), or

simultaneously, the mqo gene which encodes malate-quinone oxidoreductase (Molenaar et al., European Journal of Biochemistry 254, 395-403 (1998)), or

the lysE gene which encodes lysine export (DE-A-195 48 222).

Furthermore, for the production of L-glutamate/L-glutamic acid, for example, one or more genes selected from the following group can be simultaneously enhanced, and in particular can be over-expressed or amplified:

the gdh gene which encodes glutamate-dehydrogenase (DE: 19907347.3), and/or

the pyc gene which encodes pyruvate carboxylase (Peters-Wendisch et al.(1998), Microbiology 144: 915-927).

Moreover, for the production of L-lysine it may be advantageous if, in addition to the enhancement of the gpsA in gene:

the pck gene which encodes phosphoenol pyruvate carboxykinase (DE 199 50 409.1, DSM 13047) and/or

the pg1 gene which encodes glucose-6-phosphate isomerase (U.S. 09/396,478, DSM 12969) is attenuated.

Furthermore, for the production of L-glutamate/L-glutamic acid it may be advantageous if, in addition to the enhancement of the gpsA gene:

the odha gene which encodes a-ketoglutarate dehydrogenase (WO 9534672 Al 951221*), or

the dtsR1 gene which encodes DtsR1 protein (WO 952324 A1 950831*), or

the dtsR2 gene which encodes DtsR2 protein (WO 9902692A A1 990121*), is simultaneously attenuated.

Moreover, for the production of amino acids, particularly L-lysine and L-glutamate/L-glutamic acid, it may be advantageous if, in addition to the over-expression of the gpsA gene, unwanted secondary reactions are suppressed (Nakayama: “Breeding of Amino Acid Producing Micro-organisms”, in: Overproduction of Microbial Products, Krumphanzl, Sikyta, Vanek (Eds.), Academic Press, London, UK, 1982).

The microorganisms which are produced according to the invention can be cultivated batch-wise or continuously, e.g. by a batch process (batch cultivation) or by a fed batch process (feed process) or by a repeated fed batch process (repetitive feed process), for the purpose of producing amino acids, particularly L-glutamate/L-glutamic acid. A review of known methods of cultivation is given in the textbook by Chmiel (Bioprozesstechnik 1. Einfluhrung in die Bioverfahrenstechnik (Gustav Fischer Verlag, Stuttgart, 1991)) and in the textbook by Storhas (Bioreaktoren und periphere Einrichtungen (Vieweg Verlag, Brunswick/Wiesbaden, 1994)).

The culture medium which is used must fulfil the requirements of the strains concerned in a suitable manner. Descriptions of culture media for various microorganisms are given in the Handbook “Manual of Methods for General Bacteriology” published by the American Society for Bacteriology (Washington D.C., USA, 1981). Suitable sources of carbon include sugar and carbohydrates such as glucose, saccharose, lactose, fructose, maltose, molasses, starch and cellulose, oils and fats such as soya oil, sunflower oil, peanut oil and cocoa fat, fatty acids such as palmitic acid, stearic acid and linoleic acid, alcohols such as glycerol and ethanol, and organic acids such as acetic acid. These substances can be used individually or in admixture. Suitable sources of nitrogen include compounds which contain organic nitrogen, such as peptone, yeast extract, meat extract, malt extract, corn steep liquor, soya bean flour and urea, and inorganic compounds such as ammonium sulphate, ammonium chloride, ammonium phosphate, ammonium carbonate and ammonium nitrate. These sources of nitrogen can be used individually or in admixture. Phosphoric acid, potassium dihydrogen phosphate or dipotassium hydrogen phosphate, or the corresponding sodium-containing salts, can be used as sources of phosphorus. In addition, the culture medium must contain salts of metals such as magnesium sulphate or iron sulphate which are necessary for growth. Finally, essential growth promoting substances such as amino acids and vitamins can be used in addition to the aforementioned substances. Moreover, suitable precursors can be added to the culture medium. The aforementioned substances which are used can be added to the culture in the form of a single batch or can be supplied in a suitable manner during cultivation.

Basic compounds such as sodium hydroxide, potassium hydroxide, ammonia or aqueous ammonia, or acidic compounds such as phosphoric acid or sulphuric acid are used in a suitable manner in order to control the pH of the culture. Anti-foaming agents such polyglycol esters of fatty acids can be used to control the generation of foam. In order to maintain the stability of plasmids, suitable substances with a selective action, such as antibiotics, can be added to the medium. In order to maintain aerobic conditions, oxygen or oxygen-containing gas mixtures such as air are passed into the culture. The temperature of the culture normally ranges from 20° C. to 45° C. and is preferably 25° C. to 40° C. Cultivation is continued until a maximum of glutamate has been formed. This target is normally reached within 10 hours to 160 hours.

The following microorganism has been deposited in the German Collection of Microorganisms and Cell Cultures (DSMZ, Brunswick, Germany) in accordance with the Budapest Convention:

Corynebacterium glutamicum strain DSM5715/pJC1gpsA as DSM 13493.

The process according to the invention can be employed for the fermentative production of amino acids, particularly L-lysine and L-glutamate/L-glutamic acid.

Legends to the FIGURE:

FIG. 1: Map of the plasmid pJC1gpsA

The numbers of base pairs are given as approximate values which can be obtained within the limits of reproducibility. The abbreviations and descriptions used have the following meanings:

| | |
|---|---|
| Orf2, rep | plasmid-coded replication origin C. glutamicum (of |
| | pHM1519) |
| gpsA: | gpsA (glycerol-3-phosphate dehydrogenase) gene from |
| | C. glutamicum ATCC13032 |
| Kan: | kanamycin-resistant gene |
| XbaI: | cleavage site of the restriction enzyme XbaI |
| PstI: | cleavage site of the restriction enzyme PstI |
| XhoI: | cleavage site of the restriction enzyme XhoI |
| SmaI: | cleavage site of the restriction enzyme SmaI |
| BglII: | cleavage site of the restriction enzyme BglII |
| EcoRI: | cleavage site of the restriction enzyme EcoRI |
| BamHI: | cleavage site of the restriction enzyme BamHI. |

### EXAMPLES

The present invention is described in more detail below with reference to examples of embodiments.

### Example 1

Production of a genomic cosmid gene bank from Corynebacterium glutamicum ATCC 13032

Chromosomal DNA from Corynebacterium glutamicum ATCC 13032 was isolated as described by Tauch et al. (1995, Plasmid 33:168-179) and was partially cleaved with the restriction enzyme Sau3AI (Amersham Pharmacia, Freiburg, Germany, product description Sau3AI, Code no. 27-0913-02). The DNA fragments were dephosphorylated with shrimp alkaline phosphatase (Roche Molecular Biochemicals, Mannheim, Germany, product description SAP, Code no. 1758250). The DNA of the cosmid vector SuperCos1 (Wahl et al. (1987) Proceedings of the National Academy of Sciences USA 84:2160-2164), purchased from Stratagene (La Jolla, USA, product description SuperCos1 cosmid vector Kit, Code no. 251301) was cleaved with the restriction enzyme XbaI (Amersham Pharmacia, Freiburg, Germany, product description XbaI, Code no. 27-0948-02) and was likewise dephosphorylated with shrimp alkaline phosphatase. The cosmid DNA was subsequently cleaved with the restriction enzyme BamHI (Amersham Pharmacia, Freiburg, Germany, product description BamHI, Code no. 27-0868-04). The cosmid DNA which was treated in this manner was mixed with the treated ATCC 13032-DNA and the batch was treated with T4 DNA ligase (Amersham Pharmacia, Freiburg, Germany, product description T4-DNA-Ligase, Code no.27-0870-04). The ligation mix was subsequently packed in phages with the aid of Gigapack II XL Packing Extract (Stratagene, La Jolla, USA, product description Gigapack II XL Packing Extract, Code no. 200217). In order to infect the E. coli strain NM554 (Raleigh et al. 1988, Nucleic Acid Research 30 16:1563-l575), the cells were taken up in 10 mM MgSO₄ and were mixed with an aliquot of the phage suspension. Infection and titration of the cosmid bank were effected as described by Sambrook et al. (1989, Molecular Cloning: A Laboratory Manual, Cold Spring Harbor), with the cells being plated out on to LB 35 agar (Lennox, 1955, Virology, 1:190) with 100 mg/l ampicillin. After incubation overnight at 37° C., recombinant individual clones were selected.

### Examples 2

Isolation and sequencing of the gpsA gene

The cosmid DNA of a single colony was isolated using a Qiaprep Spin Miniprep Kit (Product No. 27106, Qiagen, Hilden, Germany) in accordance with the manufacturer's instructions and was partially cleaved with the restriction enzyme Sau3AI (Amersham Pharmacia, Freiburg, Germany, product description Sau3AI, Product No. 27-0913-02). The DNA fragments were dephosphorylated with shrimp alkaline phosphatase (Roche Molecular Biochemicals, Mannheim, Germany, product description SAP, Product No. 1758250). After separation by gel electrophoresis, cosmid fragments of the order of 1500 to 2000 bp were isolated using a QiaExII Gel Extraction Kit (Product No. 20021, Qiagen, Hilden, Germany). The DNA of the sequencing vector, pZero-1, purchased from Invitrogen (Groningen, Holland, product description Zero Background Cloning Kit, Product No. K2500-01) was cleaved with the restriction enzyme BamHI (Amersham Pharmacia, Freiburg, Germany, product description BamHI, Product No. 27-0868-04). Ligation of the cosmid fragments in the sequencing vector pZero-1 was effected as described by Sambrook et al. (1989, Molecular Cloning: A laboratory Manual, Cold Spring Harbor), the DNA mixture being incubated overnight with T4 ligase (Pharmacia Biotech, Freiburg, Germany). This ligation mix was subsequently transferred into E. coli strain DH5αAMCR (Grant, 1990, Proceedings of the National Academy of Sciences U.S.A., 87:4645-4649) by means of electroporation (Tauch et al. 1994, FEMS Microbiol Letters, 123:343-7) and the batch was plated out on LB agar (Lennox, 1955, Virology, 1:190) with 50 mg/l zeocin. The plasmid was prepared from the recombinant clone using a Biorobot 9600 (Product No. 900200, Qiagen, Hilden, Germany). Sequencing was effected by the dideoxy chain termination method of Sanger et al. (1977, Proceedings of the National Academy of Sciences U.S.A., 74:5463-5467) with modifications according to Zimmermann et al. (1990, Nucleic Acids Research, 18:1067). The “RR d-Rhodamine Terminator Cycle Sequencing Kit” of PE Applied Biosystems (Product No. 403044, Weiterstadt, Germany) was used for this purpose. Separation by gel electrophoresis and analysis of the sequencing reaction were effected in a “rotiphoresis NF acrylamide/bisacrylamide” gel (29:1) (Product No. A124.1, Roth, Karlsruhe, Germany), using the “ABI Prism 377” sequencing device of PE Applied Biosystems (Weiterstadt, Germany).

The raw sequence data which were obtained were subsequently processed using the Staden software package (1986, Nucleic Acids Research, 14:217-231) Version 97-0. The individual sequences of the pzerol derivatives were assembled to form a coherent contiguous sequence. Computer-aided analysis was performed using the XNIP program (Staden, 1986, Nucleic Acids Research, 14:217-231). Further analyses were performed using “BLAST search programs” (Altschul et al., 1997, Nucleic Acids Research, 25:3389-3402), compared with the non-redundant databank of the National Center for Biotechnology Information (NCBI, Bethesda, Md., USA).

The nucleotide sequence obtained is illustrated in SEQ ID No. 1. Analysis of the nucleotide sequence showed the presence of an open reading frame comprising 996 base pairs, which was designated as the gpsA gene. The gpsA gene encodes a protein comprising 332 amino acids (SEQ ID No.2).

### Example 3

Cloning the gpsA gene in the vector pJC1

Chromosomal DNA from Corynebacterium glutamincum ATCC 13032 was isolated as described by Tauch et al. (1995, Plasmid 33:168-179). A DNA fragment bearing the gpsA gene was amplified by polymerase chain reaction. The following primers were used for this purpose:

5′-TGC TCT AGA TGC GGG TGG CTT GGG ACA T-3 ′(SEQ ID No. 3)

5′-TGC TCT AGA ACG ACT GCG ACG CGG ACT TTT C-3 ′(SEQ ID No. 4)

The primers illustrated were synthesised by MWG Biotech (Ebersberg, Germany) and the PCR reaction was carried out by the standard PCR method of Innis et al.(PCR protocol. A guide to methods and applications, 1990, Academic Press). The primers enabled amplification to be effected of a DNA fragment with a size of about 1193 bp and bearing the gpsA gene of Corynebacterium glutamicum.

After separation by gel electrophoresis, the PCR fragment was isolated from the agarose gel using a QiaExII Gel Extraction Kit (Product No. 20021, Qiagen, Hilden, Germany).

The E. coli-C. glutamicum shuttle vector pJC1 (Cremer et al., 1990, Molecular and General Genetics 220: 478 - 480) was used as a vector. This plasmid was completely cleaved with the restriction enzyme BamHI, was treated with Klenow polymerase (Roche Diagnostics GmbH, Mannheim, Germany) and was subsequently dephosphorylated with shrimp alkaline phosphatase (Roche Diagnostics GmbH, Mannheim, Germany, product description SAP, Product No. 1758250).

The gpsA fragment obtained in this manner was mixed with the prepared vector pJC1 and was ligated with the aid of a SureClone Ligation Kit (Amersham Pharmacia Biotech, Uppsala, Sweden) according to the manufacturer's instructions. The ligation batch was transformed in the E. coli strain DH5α(Hanahan, in: DNA cloning. A practical approach. Vol. I. IRL Press, Oxford, Washington DC, USA). Plasmid-bearing cells were selected by plating out the transformation batch on LB agar (Lennox, 1955, Virology, 1:190) with 50 mg/l kanamycin. After incubation overnight at 37° C., recombinant individual clones were selected. Plasmid DNA was isolated from a transformant using a Qiaprep Spin Miniprep Kit (Product No. 27106, Qiagen, Hilden, Germany) according to the manufacturer's instructions and was cleaved with the restriction enzyme XbaI in order to investigate the plasmid by subsequent agarose gel electrophoresis. The plasmid obtained was designated as pJC1gpsA.

### Example 4

Transformation of the strains ATCC13032 and DSM5715 with the plasmid pJC1gpsA

The C. glutamicum strains ATCC13032 and DSM5715 were transformed with the plasmid pJC1gpsA using the electroporation method described by Liebl et al. (FEMS Microbiology Letters, 53:299-303 (1989)). The transformants were selected on LBHIS agar consisting of 18.5 g/l brain-heart infusion bouillon, 0.5 M sorbitol, 5 g/l bacteriological trypton, 2.5 g/l bacteriological yeast extract, 5 g/l NaCl and 18 g/l bacteriological agar which was supplemented with 25 mg/l kanamycin. Incubation was effected for 2 days at 33° C.

Plasmid DNA was isolated from each transformant by the usual methods (Peters-Wendisch et al., 1998, Microbiology, 144, 915-927), was cut with the restriction endonuclease XbaI and the plasmid was investigated by subsequent agarose gel electrophoresis. The strains obtained were designated as ATCC13032/pJC1gpsA and DSM5715/pJC1gpsA.

### Example 5

Production of L-glutamate/L-glutamic acid using the strain ATCC13032/ pJC1gpsA The C. glutamicum strain ATCC13032/pJC1gpsA which was obtained in Example 4 was cultivated in a nutrient medium suitable for the production of glutamate, and the glutamate content in the culture supernatant was determined.

For this purpose, the strain was first incubated on an agar plate with the corresponding antibiotic (brain-heart agar with kanamycin (50 mg/l)) for 24 hours at 33° C. A preliminary culture was inoculated with this agar plate culture (10 ml medium in an 100 ml Erlenmeyer flask). The complete medium CgIII (2.5 g/l NaCl, 10 g/l bacteriological peptone, 10 g/l bacteriological yeast extract, pH 7.4, 20 g/l glucose (autoclaved separately)) was used as the medium for the preliminary culture. Kanamycin (25 mg/l) was added to the latter. The preliminary culture was incubated for 16 hours at 33° C., at 240 rpm on a shaker. A main culture was inoculated with this preliminary culture so that the initial OD (660nm) of the main culture was 0.1. The medium CgXII was used for the main culture.

After preliminary cultivation in CgIII medium (Keilhauer et al. 1993, Journal of Bacteriology 175:5595-5603), the strain ATCC13032/pJC1gpsA was cultivated in CgXII production medium (Keilhauer et al. 1993, Journal of Bacteriology 175:5595-15=5603). 4% glucose and 50 mg/l kanamycin sulphate were added.

To induce glutamate formation, 20 g Tween 60 (P-1629, Sigma-Aldrich, Deisenhofen, Germany) plus 80 ml water were mixed and autoclaved. About 4 hours after inoculation, 75 μl of this Tween solution was added to the culture and cultivation was continued.

Cultivation was effected in a volume of 10 ml in a 100 ml Erlenmeyer flask fitted with baffles. Kanamycin (25 mg/l) was added. Cultivation was conducted at 33° C. and 80% atmospheric humidity.

After 48 hours, the OD was determined at a measuring wavelength of 660 nm using a Biomek 1000 (Beckmann Instruments GmbH, Munich). The quantity of glutamate formed was determined using an amino acid analyser supplied by Eppendorf-BioTronik (Hamburg, Germany), by ion exchange chromatography and subsequent derivative formation using ninhydrin as a detector.

The results of the experiment are given in Table 1.

TABLE 1

| | OD (660 | Glutamate-HCl |
|---|---|---|
| Strain | nm) | mM |
| ATCC13032/pJC1gpsA | 14.7 | 98 |
| ATCC13032 | 13.8 | 94 |

### Example 6

Production of L-lysine

The C. glutamicum strain DSM5715/pJC1gpsA obtained in Example 4 was cultivated in a nutrient medium suitable for the production of lysine, and the lysine content in the culture supernatant was determined.

For this purpose, the strain was first incubated on an agar plate with the corresponding antibiotic (brain-heart agar with kanamycin (50 mg/l)) for 24 hours at 33° C. A preliminary culture was inoculated with this agar plate culture (10 ml medium in an 100 ml Erlenmeyer flask). The complete medium CgIII (2.5 g/l NaCl, 10 g/l bacteriological peptone, 10 g/l bacteriological yeast extract, pH 7.4, 20 g/l glucose (autoclaved separately)) was used as the medium for the preliminary culture. Kanamycin (25 mg/l) was added thereto. The preliminary culture was incubated for 16 hours at 33° C., at 240 rpm on a shaker. A main culture was inoculated with this preliminary culture so that the initial OD (660nm) of the main culture was 0.1. The medium MM was used for the main culture.

Medium MM

| | | |
|---|---|---|
| CSL (corn steep liquor) | 5 | g/l |
| MOPS (morpholinopropanesulphonic | 20 | g/l |
| acid) | | |
| glucose (autoclaved separately) | 50 | g/l |
| (NH₄)₂SO₄ | 25 | g/l |
| KH₂PO₄ | 0.1 | g/l |
| MgSO₄ * 7 H₂O | 1.0 | g/l |
| CaCl₂ * 2 H₂O | 10 | mg/l |
| FeSO₄ * 7 H₂O | 10 | mg/l |
| MnSO₄ * H₂O | 5.0 | mg/l |
| Biotin (filtered under sterile | 0.3 | mg/l |
| conditions) | | |
| thiamine * HCl (filtered under | 0.2 | mg/l |
| sterile conditions) | | |
| L-leucine | 0.1 | g/l |
| CaCO₃ | 25 | g/l |

The CSL, the MOPS and the salt solution were adjusted to pH 7 with aqueous ammonia and were autoclaved. The sterile substrate and vitamin solutions were then added, together with dried, autoclaved CaCO₃.

Cultivation was effected in a volume of 10 ml in a 100 ml Erlenmeyer flask fitted with baffles. Kanamycin (25 mg/l) was added. Cultivation was conducted at 33° C. and 80% atmospheric humidity.

After 72 hours, the OD was determined at a measuring wavelength of 660 nm using a Biomek 1000 (Beckmann Instruments GmbH, Munich). The quantity of lysine formed was determined using an amino acid analyser supplied by Eppendorf-BioTronik (Hamburg, Germany), by ion exchange chromatography and subsequent derivative formation using ninhydrin as a detector.

The results of the experiment are given in Table 2:

TABLE 2

| | OD (660 | Lysine-HCl |
|---|---|---|
| Strain | nm) | g/l |
| DSM5715/pJC1gpsA | 7.3 | 14.8 |
| DSM5715 | 7.6 | 13.5 |

### Modifications and other embodiments

Various modifications and variations of the described products, compositions and methods as well as the concept of the invention will be apparent to those skilled in the art without departing from the scope and spirit of the invention. Although the invention has been described in connection with specific preferred embodiments, it should be understood that the invention as claimed is not intended to be limited to such specific embodiments. Various modifications of the described modes for carrying out the invention which are obvious to those skilled in the molecular biological, biological, chemical or related fields are intended to be within the scope of the following claims.

### Incorporated by reference

Each document, patent application or patent publication cited by or referred to in this disclosure is incorporated by reference in its entirety. Any patent document to which this application claims priority is also incorporated by reference in its entirety. Specifically, German priority document DE100.32 174.7, filed Jul. 1, 2000 is hereby incorporated by reference.

1. A polynucleotide, that encodes the amino acid sequence of SEQ ID NO: 2, a variant thereof which encodes a protein that is at least 70% homologous to SEQ ID NO: 2, or a variant thereof that hydridizes to SEQ ID NO: 1 under stringent conditions.

2. The polynucleotide of claim 1, that encodes a protein having glycerol-3-phosphate activity.

3. The polynucleotide of claim 2, wherein said polynucleotide hybridizes to SEQ ID NO: 1 under stringent conditions.

4. The polynucleotide of claim 2, wherein said polynucleotide is at least 70% homologous to SEQ ID NO: 1.

5. A polynucleotide comprising at least 15 consecutive nucleotides of SEQ ID NO: 1 or at least 15 consecutive nucleotides of the complement of SEQ ID NO: 1.

6. A polynucleotide which is complementary to the polynucleotide of claim 1.

7. A replicable nucleic acid comprising the polynucleotide of claim 1.

8. The replicable nucleic acid of claim 7, which is a plasmid or phage vector.

9. A host cell comprising the polynucleotide sequence of claim 1.

10. A coryneform bacterium comprising a polynucleotide which encodes an enhanced amount of the polypeptide comprising the amino acid sequence of SEQ ID NO: 2, or which encodes a polypeptide variant of the amino acid sequence of SEQ ID NO: 2 that has glycerol-3-phosphate dehydrogenase activity.

11. The coryneform bacterium of claim 10, that overexpresses a polypeptide having glycerol-3-phosphate dehydrogenase activity.

12. The coryneform bacterium of claim 10, wherein said polynucleotide sequence is present at an enhanced copy number.

13. The coryneform bacterium of claim 10, that expresses a polypeptide having enhanced glycerol-3-phosphate dehydrogenase activity.

14. The coryneform bacterium of claim 10, selected from the group consisting of Corynebacterium glutamicum (ATCC13032), Corynebacterium acetoglutamicum (ATCC15806), Corynebacterium acetoacidophilum (ATCC13870), Corynebacterium thermoaminogenes (FERM BP-1539), Corynebacterium melassecola (ATCC17965), Brevibacterium flavum (ATCC14067), Brevibacterium lactofermentum (ATCC13869), Brevibacterium divaricatum (ATCC14020), Corynebacterium glutamicum FERM-P 1709, Brevibacterium flavum FERM-P 1708, Brevibacterium lactofermentum FERM-P 1712, Corynebacterium glutamicum FERM-P 6463, Corynebacterium glutamicum FERM-P 6464 and Corynebacterium glutamicum DSM5715.

15. The coryneform bacterium of claim 10, further comprising an element which enhances expression of the polynucleotide sequence encoding glycerol-3-phosphate dehydrogenase, or a polynucleotide sequence encoding a variant thereof that has glycerol-3-phosphate dehydrogenase activity, selected from the group consisting of a promoter, an inducible promoter, a regulatory region, a ribosome binding site, an expression cassette, an element extends the life of mRNA, and an element that prevents the degradation of an expressed protein.

16. A coryneform bacterium according to claim 10, wherein said bacterium is transformed by a plasmid vector comprising a nucleotide sequence encoding the polypeptide of SEQ ID NO: 2 or a variant thereof having glycerol-3-phosphate dehydrogenase activity.

17. A coryneform bacterium according to claim 10, wherein said bacterium is Corynebacterium glutamicum DSM 13493 or a mutant thereof.

18. A method for making glycerol-3-phosphate dehydrogenase, or a variant thereof that has glycerol-3-phosphate dehydrogenase activity, comprising: culturing the host cell of claim 9 for a time and under conditions suitable for expression of glycerol-3-phosphate dehydrogenase or said variant, and obtaining the glycerol-3-phosphate dehydrogenase or said variant thereof having glycerol-3-phosphate dehydrogenase activity.

19. A method for making glycerol-3-phosphate dehydrogenase, or a variant thereof that has glycerol-3-phosphate dehydrogenase activity, comprising: culturing the corynebacterium of claim 10 for a time and under conditions suitable for expression of glycerol-3-phosphate dehydrogenase or said variant, and obtaining the glycerol-3-phosphate dehydrogenase or said variant thereof having glycerol-3-phosphate dehydrogenase activity.

20. Isolated glycerol-3-phosphate dehydrogenase encoded by the nucleotide sequence of claim 1, or fragment thereof that has glycerol-3-phosphate activity.

21. A process for fermentive production of an L-amino acid, comprising culturing a corynebacterium of Claim 10 under conditions suitable for production of an L-amino acid, and recovering the L-amino acid from the culture medium or from said corynebacterium.

22. The process of claim 21, wherein said corynebacterium lacks at least one metabolic pathway which reduces the formation of the L-amino acid.

23. The process of claim 21, wherein said L-amino acid is L-glutamate/L-glutamic acid or L-lysine.

24. The process of claim 21, for the production of L-glutamate/L-glutamic acid in which said corynebacterium comprises an enhanced, amplified or over-expressed gene selected from the group consisting of:
the dapA gene which encodes dihydrodipicolinatesynthase,
the dapE gene which encodes succinyldiaminopimelate desuccinylase,
the lysC gene which encodes a feedback-resistant aspartate kinase,
the tpi gene which encodes triose phosphate isomerase,
the gap gene which encodes glyceraldehyde-3-phosphate dehydrogenase,
the pgk gene which encodes 3-phosphoglycerate kinase,
the pyc gene which encodes pyruvate carboxylase,
the mqo gene which encodes malate:quinone oxidoreductase,
the lysE gene which encodes lysine export.

25. The process of claim 21, for the production of L-lysine, wherein said corynebacteria comprises an attenuated:
a) pck gene which encodes phosphoenol pyruvate carboxykinase,or
b) the pgi gene which encodes for glucose-6-phosphate isomerase.

26. A method for detecting a nucleic acid with at least 70% homology to nucleotide of claim 1, comprising contacting a nucleic acid sample with a probe or primer comprising at least 15 consecutive nucleotides of the nucleotide sequence of claim 1, or at least 15 consecutive nucleotides of the complement thereof.

27. A method for producing a nucleic acid with at least 70% homology to nucleotide of claim 1, comprising contacting a nucleic acid sample with a primer comprising at least 15 consecutive nucleotides of the nucleotide sequence of claim 1, or at least 15 consecutive nucleotides of the complement thereof.


SEQUENCE LISTING (4 sequences)

SEQ ID NO: 1 - DNA, 1416 bp, Corynebacterium glutamicum
  CDS (211)..(1206)
acataagtgaatgaaaaactacttccatctattgttcaccagcgacccgctcattgcacattctggactcggcgtgtggcgacatttttggatgattcctggcaaattctgggcagcagcggcaggtttccaggaggtttccatgcgggtggcttgggacatgggctaacctgagacggttaaatatcgttttcgaaaggtgggtttcgcgtggtttctgtaagcgtgatgggtgcaggttcctggggaaccacgttggccaaggtcttctctgatgctggcaacgctgtgacgttgtgggcgaggcgggaagagttggcaagcaccatccgtgacagccatgaaaaccgtgattaccttccggggattacgttgccggagtcgctgcaggtcacatcatcggcaacggaggctttagagggcgcagccattgtggtgttggcgattccttcgcaggcgttgcgtggcaatttggcggagtggaaagagacgatcccgcaggatgcgaccttggtgtccttggctaaaggtattgaaaagggcacgcacctgcggatgagtgaagtgatcgcggaggtgacggaagcggatccttcacgcatcgcggtgttgtcggggccaaaccttgctcgtgagatcgcggaggggcagcctgcagctacggtgattgcttgccctgatgaaaaccgagcgaaacttgtgcaggctgcagtggctgcgccgtatttccgcccgtacaccaacactgatgtggtgggcactgaaatcggtggtgcgtgtaagaacgtcatcgcgctggcctgtggtatttcccatggttacggcctgggtgagaacaccaatgcatcgttgattactcgtggccttgcagagatcgcacgcctcggtgccacattgggtgcggatgcgaagactttttctggccttgcgggaatgggcgacttggtggctacgtgttcatcaccgctgtcgcgtaaccgcagcttcggtgagcgtttgggtcagggtgaatccctagagaaggctcgcgaggcaaccaatggtcaggttgcggagggtgttatttcctcgcagtcgatttttgatcttgccaccaagcttggtgtggagatgccgatcacccaggctgtctacggtgtgtgccaccgagatatgaaagtaactgacatgattgtggctctcatgggcaggtctaagaaggctgagtagtcttaggttgtaagcttcaatgctgtgagcaactctaattctggaaaagtccgcgtcgcagtcgtttatggtggtcgcagttctgagcactccgtctcctgcgtctccgctggtgctatcatggcccatctcgatcctgagaagtacgatgtgattcccgtcggcattactgtcgacggcgcgtgggttgttggtgaaaccgatcca

SEQ ID NO: 2 - protein, 332 aa, Corynebacterium glutamicum
VVSVSVMGAGSWGTTLAKVFSDAGNAVTLWARREELASTIRDSHENRDYLPGITLPESLQVTSSATEALEGAAIVVLAIPSQALRGNLAEWKETIPQDATLVSLAKGIEKGTHLRMSEVIAEVTEADPSRIAVLSGPNLAREIAEGQPAATVIACPDENRAKLVQAAVAAPYFRPYTNTDVVGTEIGGACKNVIALACGISHGYGLGENTNASLITRGLAEIARLGATLGADAKTFSGLAGMGDLVATCSSPLSRNRSFGERLGQGESLEKAREATNGQVAEGVISSQSIFDLATKLGVEMPITQAVYGVCHRDMKVTDMIVALMGRSKKAE

SEQ ID NO: 3 - DNA, 28 bp, Artificial
  synthetic primer
tgctctagatgcgggtggcttgggacat

SEQ ID NO: 4 - DNA, 31 bp, Artificial
  synthetic primer
tgctctagaacgactgcgacgcggacttttc